(19) United States
(12) Patent Application Publication
(10) Pub. No.: US 2014/0200401 A1
(43) Pub. Date: Jul. 17, 2014

(54) POSITIONAL STABILIZATION AND SECUREMENT COUPLING ATTACHMENT FOR USE IN LAPAROSCOPIC SURGERY AND METHOD OF USE

(71) Applicant: George S. Ferzli, Staten Island, NY (US)

(72) Inventor: George S. Ferzli, Staten Island, NY (US)

(21) Appl. No.: 13/742,358

(22) Filed: Jan. 16, 2013

(51) Int. Cl. A61B 1/00

(52) U.S. Cl. CPC A61B 1/00147; A61B 1/00131
USPC 600/101

(57) ABSTRACT

A coupling attachment includes a position stabilization portion and a laparoscopic instrument restraint portion, the latter which is configured to at least partially surround a shaft or one or more other portions of a laparoscopic instrument in a manner which positionally restrains or inhibits movement of the instrument and/or a portion or portions thereof when the position stabilization portion is supportably engaged, for example, by a clamp.

### CROSS REFERENCE TO RELATED APPLICATIONS

This application claims the benefit of U.S. Provisional Application No. 61/665,319 filed Jun. 28, 2012 entitled LAPAROSCOPIC SURGERY CLAMP ATTACHMENT.

### BACKGROUND OF THE INVENTION

The invention is directed to laparoscopic equipment, and more particularly to a coupling attachment which, when used in conjunction with laparoscopic instrumentation, facilitates the surgeon in the performance of laparoscopic procedures.

In order to perform a laparoscopic procedure, a camera is used to observe the interior of the patient so that proper surgical measures can be implemented by other types of equipment inserted inside the patent. During the laparoscopic surgery, according to conventional practice, the surgeon's assistant generally holds the camera head and guides the camera in, out, to the left, to the right, or rotates it as needed. This can be a tiring process and, as is often the case, the assistant experiences fatigue after holding the otherwise unsupported camera during prolonged surgery. Additionally, the assistant is commonly at risk of obstructing the working path of the surgeon.

Several approaches have been suggested in the prior art to hold and positionally control laparoscopic instruments during surgery, in static, movable and robotic embodiments. When used in connection, for example, with laparoscopic cameras, each of these devices typically attach to the camera head. While conventional devices have addressed the need for providing mechanical assistance to the surgeon in controlling or maintaining the position of a laparoscopic camera during surgery, they are cumbersome to install, and each present potential drawbacks. For example, because of their size, conventional devices can obstruct the surgeon's working path, analogous with the problem frequently caused by a hand of an assistant. Although reusable, these existing devices are also expensive to initially purchase. They are complex to operate, requiring additional training and application steps during surgery. Expensive, reusable equipment, also requires post-surgery sterilization.

One example of such a conventional device is described in U.S. Pat. No. 5,779,623, in which a remote controlled “Positioner for Medical Instruments” is described. It is complex, reusable and an expensive solution.

In US Pat. Pub. 2006/0161136, a “Surgical Accessory Clamp and System Method” is described. Such device similarly presents an Operating Room infrastructure solution with complex and expensive solutions for robotic instrument support.

No simple solution for positionally stabilizing and/or securing laparoscopic equipment has heretofore been suggested in either a disposable version, or in a compact and reusable form.

Furthermore, many of the conventional approaches fail to meet the basic need of simplifying the surgical operation, but instead introduce more complexity, and the potential of obstruction to the surgeon.

### SUMMARY OF THE INVENTION

An object of the invention is to provide a coupling attachment that presents a relatively low cost, sterile solution to supporting and/or positionally maintaining laparoscopic surgical instruments/equipment and/or portions thereof, including, for example, laparoscopic cameras, during surgery.

The invention addresses these and other objects by providing a coupling attachment which, when engaged with a laparoscopic instrument or instruments or portions thereof, provides a convenient manner by which the laparoscopic equipment can be positionally supported or securably maintained, thereby providing a cost effective way to support the instrumentation, for example, a camera, during prolonged surgery or during times of potential interference from the camera operator with the working operation of the surgeon.

In an embodiment of the invention, the coupling attachment is engageably receivable to one or more laparoscopic surgical instruments, such as, for example, a camera, and includes a wing or other structural feature which is adapted to being securable by commonly used sterile surgical clamps (of which Ring forceps and Kelly clamps are examples). The various clamp attachment options present a convenient attachment and coupling method of the clamp to the surgical instrument.

In another embodiment, the coupling attachment is provided in the form of an elastic clip-on ring having a generally cylindrical inner bore in which a partial opening running in an axial direction of the coupling attachment in formed, which allows it to be pushed over a camera shaft, and the like. The inherent spring action in the elastically formed ring shape allows for a snap fit over the camera shaft, thereby maintaining same in removable captive engagement. This spring action is achieved through a function of geometric design, choice of material and/or manufacturing method, and by virtue of the further option of adding reinforcing stiffeners in the product during the manufacturing process. The finish to the inside of the ring, where it makes contact with the instrument shaft, will advantageously be sufficiently polished to ensure a snug fit, while also enabling rotation of the camera shaft, and axial movement of the clamped position to proximal and distal positions. This embodiment further supports the manufacturing of multiple ring sizes to accommodate leading contemporary instrument sizes, (for example, 2 mm, 5 mm, 7 mm, 10 mm, etc.) and can be easily adapted to any future size variations.

A further stabilization option exists to completely close the open end of the snap fit ring around the instrument shaft with a quick release buckle or strap fastener structural approach, that advantageously allows for easy operation.

In a particularly advantageous embodiment, the coupling attachment includes multiple position stabilization portions conveniently in the form of wings or other protrusions, that allow the coupling attachment to be grasped with a surgical clamp, for example, Ring forceps or a Kelly clamp). These clamps are present in all minor and major operating rooms, everywhere in the world. Advantageously, these protrusions should not bend excessively when the clamp is attached, for desired stability. Optional surface features on the wings (or protrusions) serving as position stabilization portions, provided in the form of ridging or other patterns, will advantageously enhance the clamp's grip.

Once clamped, the handle of the Kelly clamp or Ring forceps can rest on the operating field, thereby stabilizing the laparoscopic instrument connected thereto via the coupling attachment. Such connected arrangement would also have the ability to swing back and forth. The attached clamps can also be held by the surgical assistant, providing an alternative ergonomic handling solution, and has the potential to reduce fatigue during prolonged surgery. The surgeon would have the opportunity to attach multiple rings and clamps to the camera shaft, at both proximal and distal positions, creating a tripod support when rested onto the operation field.

The coupling attachment will advantageously be comprised of a relatively soft rubber, silicone, high-density foam material, or any other suitable material that is advantageously sterilizable if so desired, and that will not cause denting or damage to the camera or other instrument shaft, at least in regions of the coupling attachment that comes in contact therewith.

The specific nature of the material properties of each design is subject to the choice of embodiment pursued and manufacturing method employed. The coupling attachment is envisioned to optionally be disposable and affordable, offering a distinct economic benefit over existing solutions referenced in the prior art discussion above.

It is further envisioned, that an alternative embodiment of the same idea, could be achieved through creating a flat single part that folds over the shaft. This folding would be facilitated through the use of a flexible or living hinge, with clear geometric indicators allowing for the proper longitudinal alignment. The fold-over clamp attachment shares the properties of the ring embodiment, providing a rigid locked attachment for the surgical clamps, while allowing controlled movement and rotation on the camera shaft. The inner surface finish will be smooth, while the outer surface will have articulation to improve the grip area for the clamp. This embodiment allows for easy scaling of the manufacturing process to support multiple camera shaft diameters.

A further benefit derived from the coupling attachment according to the invention is that the device can be positioned to block the laparoscope against sliding into the abdomen. In some cases, efficient use may even negate the need for the surgical assistant.

In an embodiment directed to a method of using the coupling attachment according to the invention, the surgeon, or his/her assistant, can apply multiple coupling attachments to the shaft of the scope (camera) or other laparoscopic instrument. Two clamp attachments at different distances from each other may allow for the use of two different length clamps, as needed.

The option also exists to clamp the laparoscopic instrument to the operating field by using the coupling attachments according to the invention, and in doing so, further optimizing the surgical process.

It is further envisioned that a thread with a radio-opaque marker could optionally be inserted into the coupling attachment during the manufacturing process, to ensure that the coupling attachments are always accounted for, in the event that a surgeon moves to open surgery due to a emergency mid-procedure.

The above, and other objects, features and advantages of the present invention will become apparent from the following description read in conjunction with the accompanying drawings.

### BRIEF DESCRIPTION OF THE DRAWINGS

FIG. 1 is a perspective view of a first embodiment of the coupling attachment according to the invention shown received on a shaft of laparoscopic instrument;

FIG. 2 is an end view of the first embodiment of FIG. 1;

FIG. 3 is a side view of the first embodiment of FIG. 1;

FIGS. 4 and 5 are perspective views of coupling attachments according to the first embodiment, sized to accommodate different diameter instrument shafts;

FIG. 6 is a top plan view of a second embodiment of the invention;

FIG. 7 is an end view of the second embodiment;

FIG. 8 is an underside perspective view of the second embodiment;

FIGS. 9 and 10 are perspective views of coupling attachments according to the second embodiment, sized to accommodate different diameter instrument shafts;

FIG. 11A and 11B are end views depicting a third embodiment of the coupling attachment according to the invention shown in open and locked positions, respectively;

FIG. 12 is a side elevational view of a fourth embodiment according to the invention to a fold-over version which includes interlocking teeth;

FIG. 13 is a top plan view of the fourth embodiment of FIG. 12;

FIG. 14 is a perspective view of the fourth embodiment;

FIG. 15 is a perspective view of a fifth embodiment which includes interlocking teeth;

FIG. 16 is an end view of the fifth embodiment;

FIG. 17 is a side elevational view of the fifth embodiment;

FIG. 18 is a perspective view of the fifth embodiment shown received about a shaft of a laparoscopic instrument;

FIG. 19 is a perspective view of a sixth embodiment according to the invention which includes a clasp feature;

FIG. 20 is a side elevational view of the sixth embodiment;

FIG. 21 is an end view of the sixth embodiment;

FIG. 22 is a perspective view of the sixth embodiment shown received about a shaft of a laparoscopic instrument;

FIG. 23 is an explanatory view of the coupling attachment according to an embodiment of the invention received to a laparoscopic instrument shaft allowing stabilization of the instrument by a clamp resting on the operative field;

FIG. 24 is an explanatory view of a coupling attachment according to an embodiment of the invention received to a laparoscopic instrument shaft allowing stabilization of the instrument by a clamp resting on the operative field and which is also clamped to the operative field; and

FIG. 25 is an explanatory view of a coupling attachment according to an embodiment of the invention used to organize and positionally maintain multiple portions of laparoscopic equipment.

### DETAILED DESCRIPTION OF THE PREFERRED EMBODIMENT

Before describing various depicted examples of different embodiments of the invention which will serve to illustrate, but not unnecessarily limit, the many ways in which the invention can be practiced without departure from the contemplated scope of the invention, the following broad considerations are outlined. In broad terms, a coupling attachment according to the invention is suitably configured to include a position stabilization portion and a laparoscopic instrument restraint portion, the latter which is configured to at least partially surround a shaft or one or more other portions of a laparoscopic instrument in a manner which positionally restrains or inhibits movement of the instrument and/or portion(s) thereof when the position stabilization portion is supportably engaged, for example, by a clamp or other form of securement. The support conveniently comprises the operating field on which, for example, a Kelly clamp, serving as the form of securement, is rested thereon (gravitational securement) or clamped thereto in addition to being clamped to the position stabilization portion of the coupling attachment (mechanical securement). Alternatively, the securement can be accomplished instead by any suitable method, for example, stitching a thread through the sheets of the operating field and through the position stabilization portion of the coupling attachment, by providing the coupling attachment with an outer sticky surface having a release tape that could peel off allowing the coupling to stick to the drapes of the operating field, etc.

For purposes herein, the term “laparoscopic instrument” or “laparoscopic equipment” is defined to include any and all devices used in laparoscopic surgery or procedures, and not exclusively the laparoscope (i.e. camera) itself). These will include, but not be limited to, portions or entireties of one or more of the following examples: a camera connected with a cord to a tower distant from the operating field, a grasper, a dissector, a retractor, a light cord cable, insufflator tubing, cautery cords connected to a power generator, ultrasonic device (eg., harmonic, ligasure) connected with a cord to the power generator, a suction irrigation device connected with two tubings to the ceiling and a collection canister, additional tubing for the regular suction, a bipolar cord, a laser cord, etc.

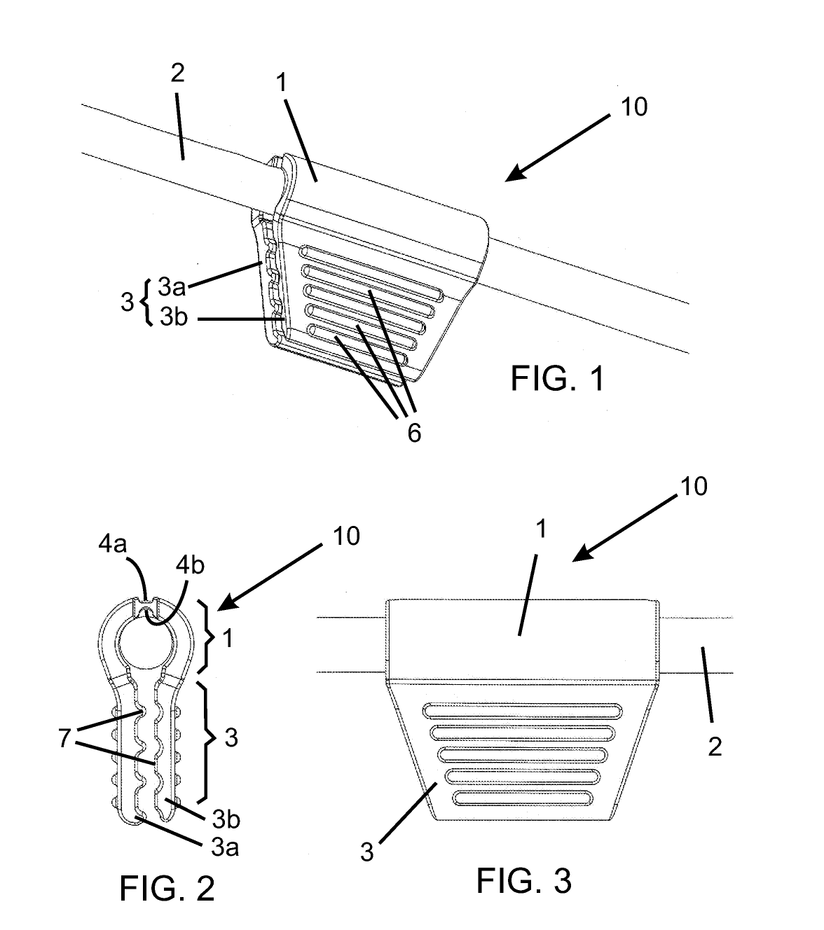

Referring now to the figures, and in particular FIGS. 1-5, a first embodiment of a coupling attachment is depicted in various views, and is generally designated by the numeral 10. Coupling attachment 10 includes a laparoscopic instrument restraint portion 1 which is configured to at least partially surround a camera shaft 2 and a position stabilization portion 3 for attachment of a securement device, for example, a conventional clamp (not shown). In the depicted example, position stabilization portion is comprised of a pair of wings 3a, 3b extending from the centrally located instrument restraint portion 1, and which are brought into facing positions when the coupling attachment 10 is received about the camera shaft 10. Coupling attachment 10 is comprised of an elastomeric material which allows at least sufficient deformation thereof to permit its installation about a camera shaft 2. While camera shaft 2 is used for illustration purposes, it will be understood that coupling attachment 10 will find utility for use with virtually any type of laparoscopic instrument designed for partial introduction into a patient's body.

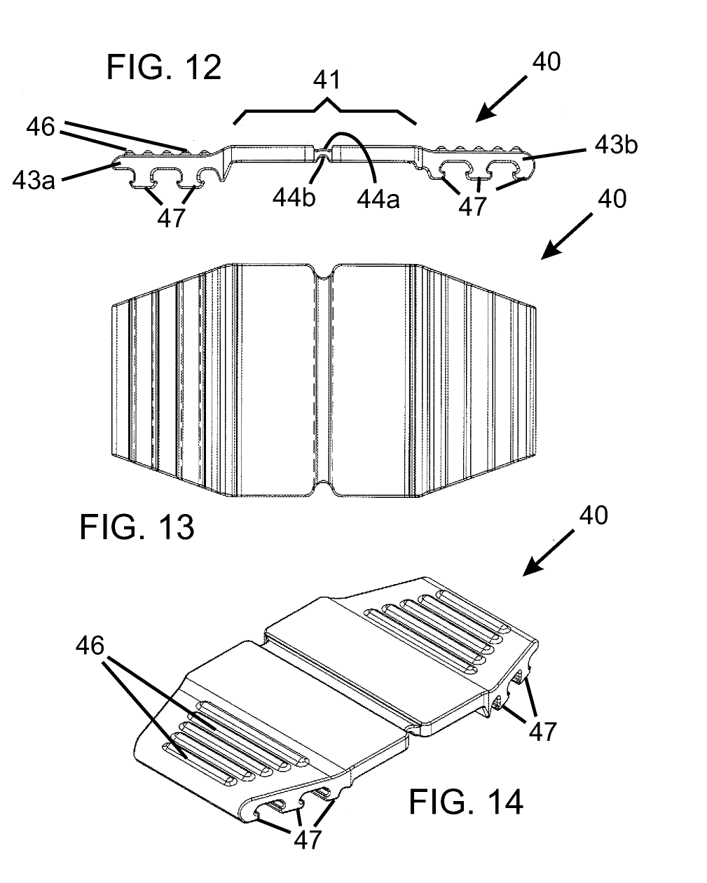

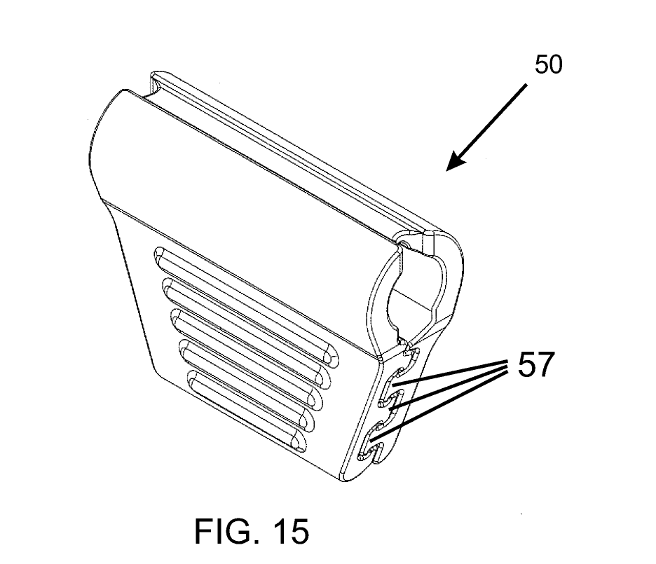

Coupling attachment 10 can be manufactured either in a generally flattened shape when unstressed (not shown in FIGS. 1-5, but can be seen in an alternative embodiment of FIGS. 12-14), in which case the coupling attachment 10 is folded around the camera shaft 2 and the pair of wings 3a, 3b then being held together by a clamp (not shown) which secures the coupling attachment 10 to the camera shaft 2, or alternatively can assume the shape as shown in FIGS. 1-5 in a resting state, in which case the pair of wings are simply spread apart to receive the camera shaft 2 within the internal receiving channel 4 of the laparoscopic instrument restraint portion 1. Particularly in the case in which coupling attachment 10 assumes a flatted shape in its natural state, axially extending indentations 4a, 4b of generally flat configuration along a center hinge line 5 are advantageously provided to allow for quick visual alignment during the folding-over of coupling attachment 10 about camera shaft 2.

Raised surface ridges 6 are advantageously provided on the outward facing surfaces of the on pair of wings 3a, 3b, operate to enhances the gripping region for the attachable clamp. Additionally, optional teeth 7 further enhance the part closure and rigidity of coupling attachment 10, while allowing for controlled axial movement or rotation along the camera shaft 2.

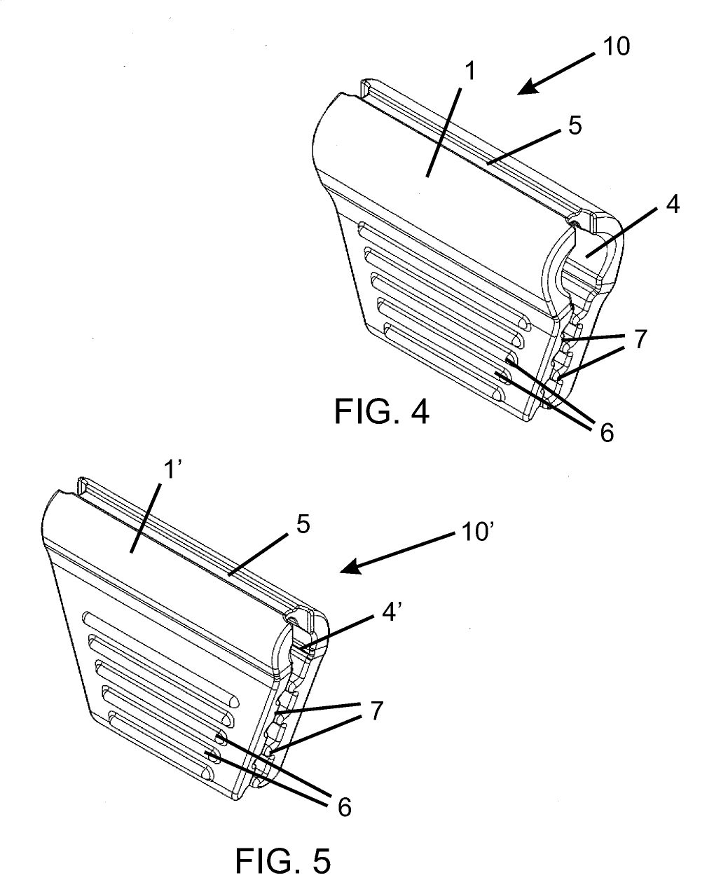

In addition to the version of coupling attachment 10 shown in FIG. 4 having an internal receiving channel 4 adapted to a shaft diameter of the camera shaft 2, as shown in FIGS. 1-3, an alternatively sized coupling attachment 10′ is shown in FIG. 5, having a internal receiving channel 4′ of smaller diameter which is configured to receive a shaft (not shown) of smaller diameter, for example, that of a laparoscopic retractor.

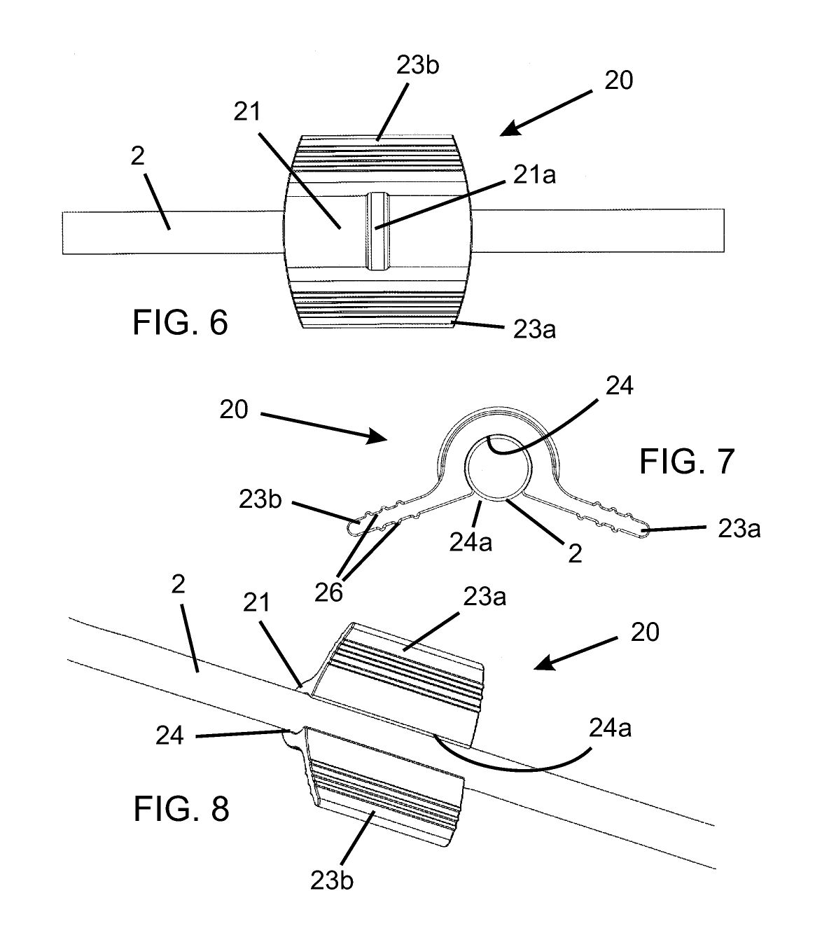

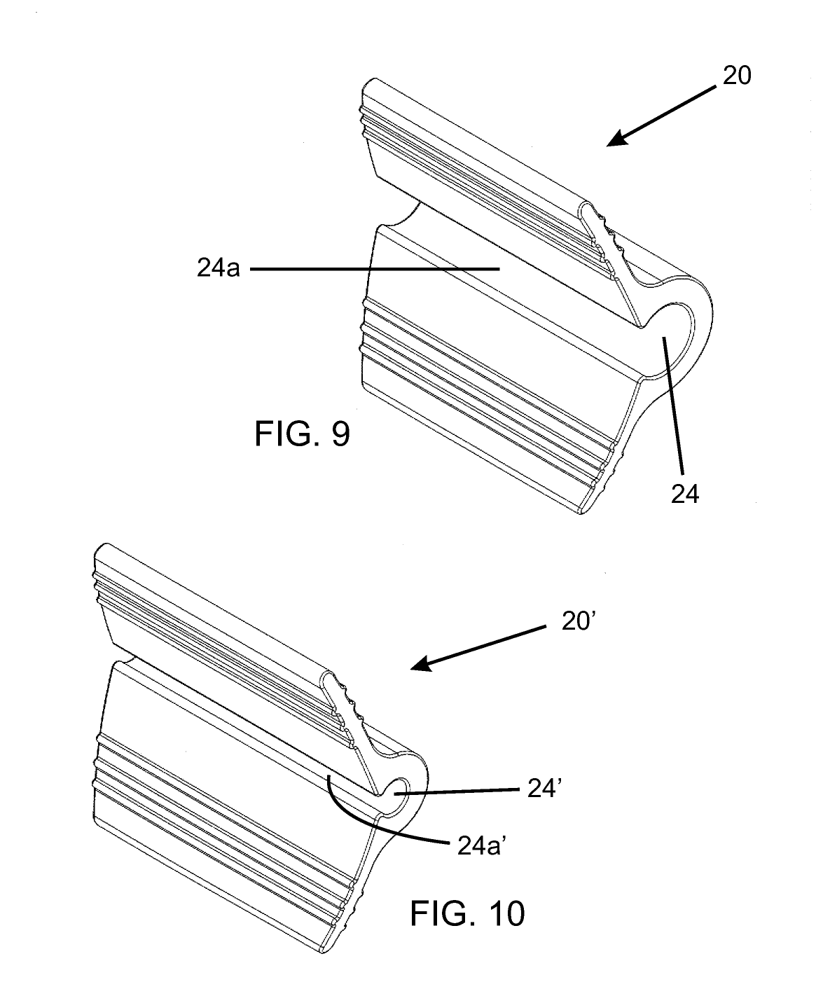

Turning now to FIGS. 6-10, a second embodiment of a coupling attachment according to the invention is depicted, generally designated by the numeral 20. Coupling attachment 20 is provided in the form of an elastic clip-on ring including a laparoscopic instrument restraint portion 21 having a generally cylindrical inner bore 24 in which a partial opening 24a running in an axial direction of the coupling attachment 20 is formed, allowing it to be pushed over a camera shaft 2. The inherent spring action attendant the elastically formed ring shape allows for a snap fit over the camera shaft 2, thereby maintaining same in removable captive engagement. This spring action is achieved through a function of geometric design, choice of material and/or manufacturing method, and the further option of adding reinforcing stiffeners in the product during the manufacturing process. The finish to the inside of the of the cylindrical inner bore 24, where it makes contact with the camera shaft 2, will advantageously be sufficiently polished to ensure a snug fit, while also enabling rotation of the camera shaft 2, and axial movement of the clamped position to proximal and distal positions. It is reiterated that this invention need not be limited to a camera shaft 2, which is only being used herein for illustration purposes.

Coupling attachment 20 includes a pair of position stabilization portions conveniently provided in the form of wings 23a, 23b which extend bilaterally from laparoscopic instrument restraint portion 21. Raised surface ridges 26 are advantageously provided on the outward facing surfaces of the pair of wings 23a, 23b, and operate to enhance the gripping region for a clamp (not shown).

FIG. 8 is a perspective view of the coupling attachment 20 clipped onto camera shaft 2. A reinforcement ridge 21a is optionally provided on the outward facing surface of the laparoscopic instrument restraint portion 21 which includes the cylindrical inner bore 24, designed to keep the attachment stable at a constant tension and position unless moved by the user.

The second embodiment also optionally allows for variations with one, three, four or more wings rather than the pair of wings 23a, 23b as shown.

The dimensions of coupling attachment 20 readily scale to support instruments with different shaft diameters, and shown, for example, in FIG. 10, in which a coupling attachment 20′ has a cylindrical inner bore 24′ suited for a smaller diameter instrument shaft, such as that of a retractor.

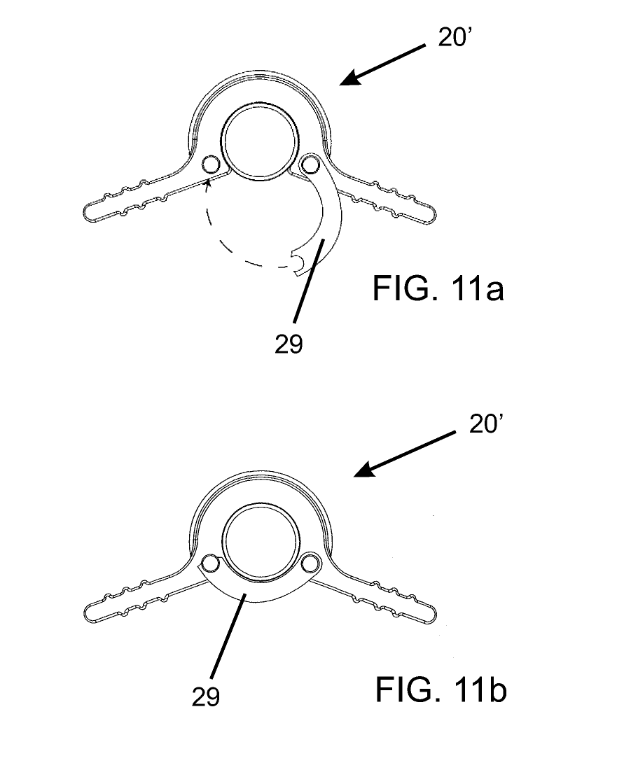

Turning now to FIGS. 11a and 11b, a thirst embodiment of a coupling attachment is depicted, which is a modified version of the second embodiment described above, and is generally designated by the numeral 20′.

As depicted in cross-sectional views, the second embodiment is modified by the optional provision of a clasp 29 which provides further stabilization for secured retention to the instrument shaft (e.g., camera shaft 2), by completely closing the open end of the snap fit ring around the camera shaft 2 which would operate as a quick release buckle or strap fastener type structural mechanism, advantageously allowing for easy operation.

Referring now to FIGS. 12-14, a fourth embodiment of a coupling attachment is depicted generally at 40. Coupling attachment 40 includes analogous structural features with the first embodiment of FIGS. 1-3, including a laparoscopic instrument restraint portion 41 which is configured to at least partially surround a shaft (not shown) and a position stabilization portion for attachment of a securement device comprised of a pair of wings 43a, 43b extending from the centrally located instrument restraint portion 41, and which are brought into facing positions when the coupling attachment 40 is received about the instrument shaft. Coupling attachment 40 is comprised of an elastomeric material which is sufficiently flexible to be conformably bent from is normally flat shape, as shown, to wrap around the instrument shaft. Axially extending indentations 44a, 44b of generally flat configuration along a center hinge line 5 are advantageously provided to allow for visual alignment during the folding-over of coupling attachment 40 about the instrument shaft.

As with the first embodiment, raised surface ridges 46 are advantageously provided on the outward facing surfaces of the on the on the pair of wings 43a, 43b, operate to enhances the gripping region for reception of the clamp.

Instead of providing the optional teeth 7 as shown with reference to the example of the first embodiment of FIGS. 1-3, the fourth embodiment as shown in FIGS. 12-14 provides the particularly advantageous feature of interlocking teeth 47 which are shaped to not just align, as in the first embodiment, but additionally to pressure fit, so that they stay interlocked, even without an attached clamp (Kelly clamp or the like). This removes the need to use a second hand to keep coupling attachment 40 in place after removing the clamp, and eases the movement axially along the instrument shaft after removal of the clamp.

The above feature is considered to be particularly advantageous since, as is often the case during surgery, the laparoscope has to be removed from the abdomen of the patient to have the lens cleansed before replacing it into the abdomen. Without the feature described above (or other alternative structural provision which maintains the closure of coupling attachment 40 about the instrument shaft and the secured engagement of the coupling attachment thereto) it is either necessary to remove the clamp, which would cause the coupling attachment to be dropped or flung onto the operating field, and later require it to be located to replace it on the shaft, or move the entire scope/coupling attachment/clamp as an attached unit, which is cumbersome and not particularly desirable. The above feature obviates these undesirable options, since upon removal of the laparoscope from the abdomen, the clamp is removed and the coupling attachment 40 remains securely in place, to be re-gripped with the clamp once the laparoscope is returned to the abdomen.

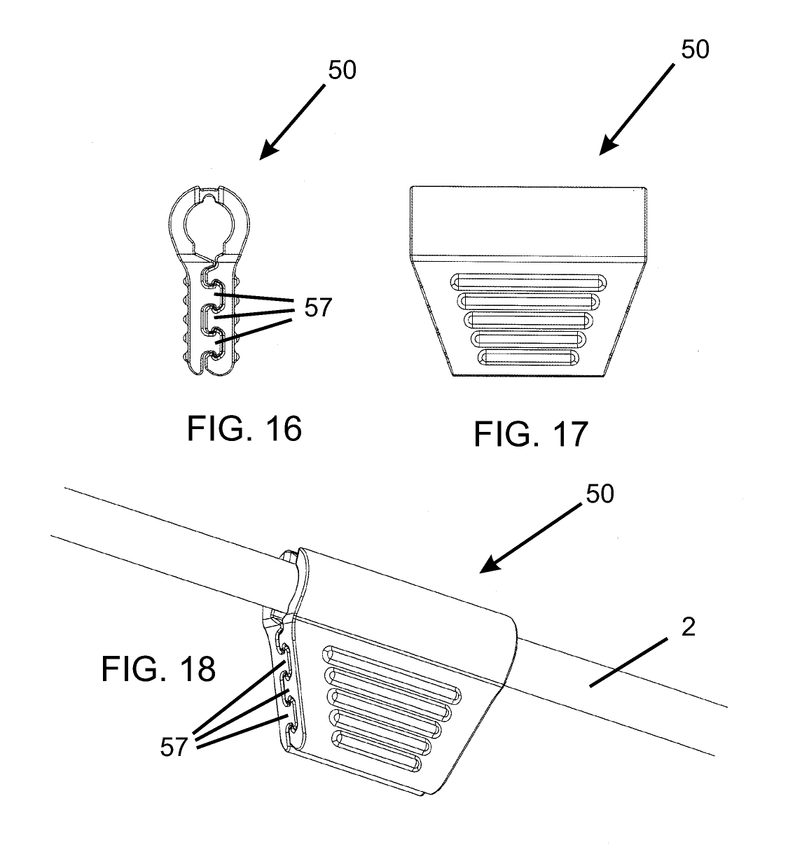

Referring to FIGS. 16-18, a fifth embodiment of a coupling attachment according to the invention is designated generally by the numeral 50. The fifth embodiment is in all respects analogous with the first embodiment of FIGS. 1-5, and as such, description their shared features is omitted as being redundant.

The fifth embodiment of FIGS. 16-18, like the fourth embodiment, replaces the optional teeth 7, as shown with reference to the example of the first embodiment of FIGS. 1-3, with interlocking teeth 57 which are shaped to not just align, as in the first embodiment, but to pressure fit so that they stay interlocked, even without an attached clamp. These provide the same advantages as those described above with reference to the fourth embodiment of FIGS. 12-14.

As discussed above, many structural approaches can be used in place of the interlocking teeth described with reference to the fourth and fifth embodiments in order to maintain secure retention of coupling attachment about the instrument shaft and achieve analogous function. One such alternative approach is shown, by example, with reference to a sixth embodiment, shown in FIGS. 16-18.

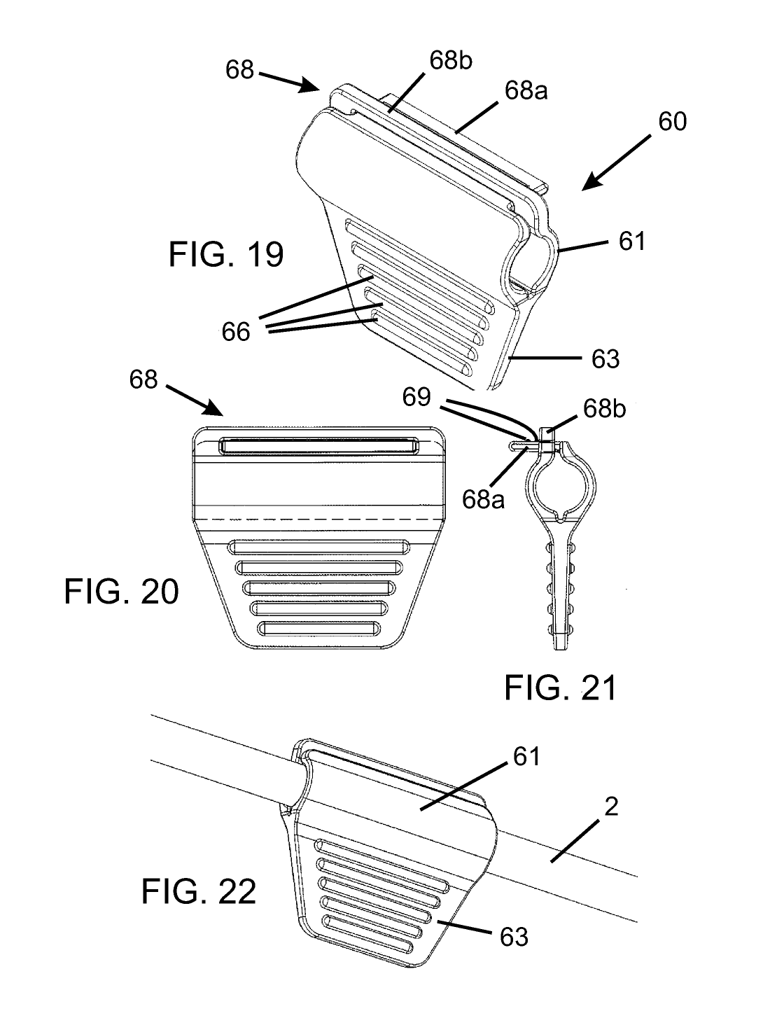

Turning to FIGS. 19-22, a coupling attachment according to the sixth embodiment is depicted, designated generally by the numeral 60. Coupling attachment 60, which like the other embodiments is comprised of a material exhibiting elastomeric properties, includes a laparoscopic instrument restraint portion 61 which is configured to at least partially surround a camera shaft 2 (see FIG. 22) and a position stabilization portion 63 for attachment of a securement device, for example, a conventional clamp (not shown). Rather than being comprised of a pair of wings as in the first embodiment, position stabilization portion is comprised a single wing 63 extending from the instrument restraint portion 61.

As with the previously described embodiments, raised surface ridges 66 are advantageously provided on the outwardly facing surfaces of the wing 63 for improved gripping by a clamp.

In the sixth embodiment, a top edge of the laparoscopic instrument restraint portion 61 is open and is modified to define a clasp mechanism 68. The clasp mechanism is comprised of a strap 68a and a slotted buckle 68b combination, in which the strap 68a is kept in place by friction through a series of ridges 69 on the outer surface of the strap 68a.

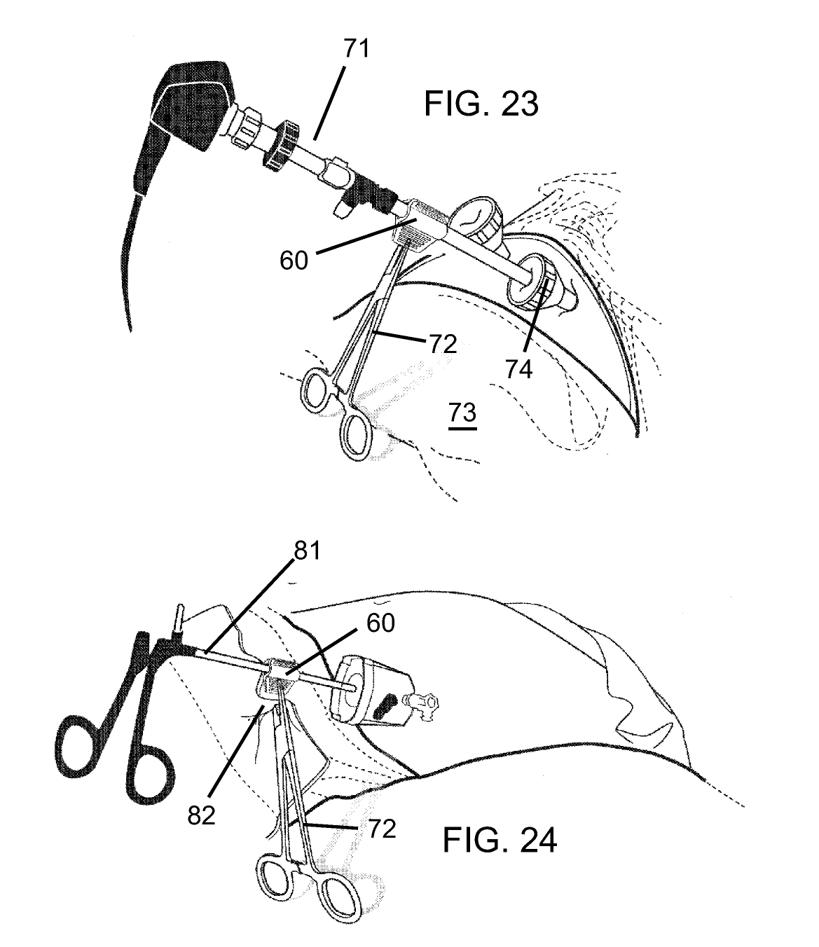

Turning now to FIGS. 23-25, various methods of use for the coupling attachment according to the invention are depicted.

FIG. 23 depicts a surgery in progress, with a laparoscopic camera 71 being supported by a Kelly clamp 72 attached to coupling attachment 60 (sixth embodiment) clipped over the camera shaft 71. The Kelly clamp 72 rests on the operating field 73. The combination of the camera 71 at its junction with trocar 74 and the handle of the Kelly clamp 74 provides gravitational support in the nature of a tripod.

FIG. 24 depicts another surgical use for the coupling attachment of the invention. As shown, in use in a laparoscopic cholecystectomy, a grasper 81 that is retracting the liver of a patient, is anchored to the operating table sterile cover using the coupling attachment 60, by placing the coupling attachment on the shaft of the grasper 81 or on the handle of the instrument, and then using a Kelly clamp 72 to grasp the coupling attachment 60 along with the surgical field drapes 82 of the operating table sterile cover. As such, the field drapes 82 serve to securely anchor the grasper 81.

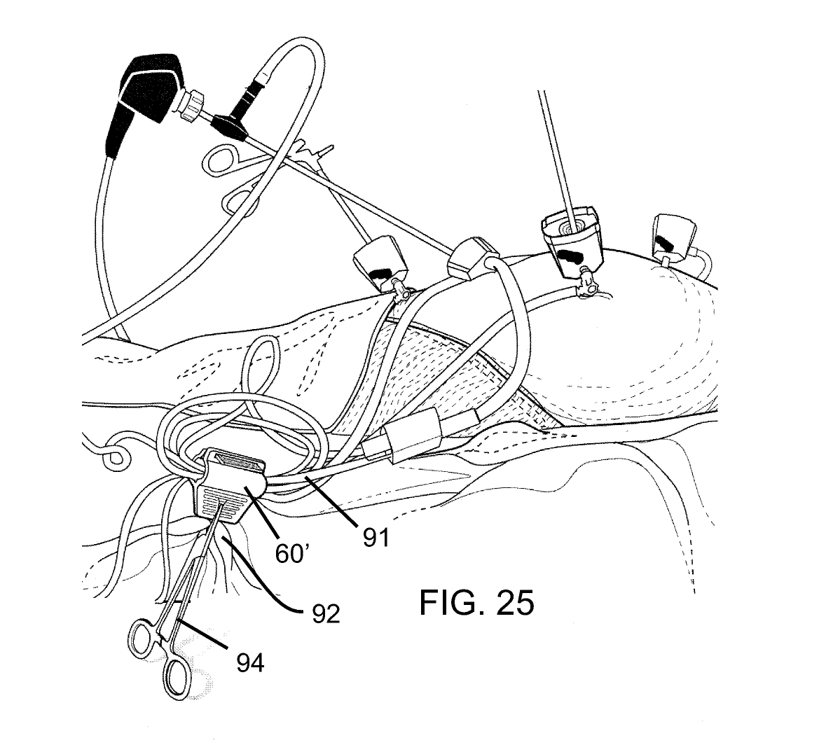

Referring to FIG. 25, a surgical procedure is illustrated in which the coupling attachment according to the invention is shown used to organize as positionally support various laparoscopic instruments and portions thereof. During laparoscopic surgery the following instruments/equipments are routinely used: a camera connected with a cord to a tower distant from the operating field, a light cord cable connected to the same tower, insufflator tubing connected to the same tower, two cautery cords connected to a power generator, an ultrasonic device (e.g., Harmonic, Ligasure) connected with a cord to the power generator, a suction irrigation device connected with two tubings to the ceiling and the collection canister, occasionally an additional tubing for the regular suction, a bipolar cord, a laser cord, etc. Consequently, there are at a general minimum, seven cords/tubings, crossing from the operating field to remote devices (power generators etc.) Ocasionally, there are sometimes even ten of them.

FIG. 25 illustrate a larger dimensioned coupling attachment around all the various cords and tubes 91 along their course. Although one is shown, several similar coupling attachments can also be used. This embodiment directed to a method of use, allows the nurse/resident/surgeon to affix cords and tubes neatly to the surgical field. The coupling attachments is shown anchored to the surgical field drapes 92 with a surgical clamp 94.

It is noted that although the coupling attachment in accordance with the invention is described as being independent of a clamp which is subsequently attached to the position stabilization portion of the coupling attachment to provide support, it is contemplated that the coupling attachment according to another embodiment, deemed within the scope of the invention, can include independent support structure integral with, or assembled to as part of, the position stabilization portion, so as to obviate the need for an external clamp separate of the coupling attachment. For example, as mentioned above, the integrated support structure can be no more than a self-sticking adhesive included on the position stabilization portion, protected by a release film which, when removed, allows adhesion to a support surface, such as the drapes of the operating field. Alternatively, the position stabilization portion can be made in a form of a modified wing which extends a sufficient distance, or extendable by an adjustable distance, from the laparoscopic instrument restraint portion, and having a suitable terminal end shape and size, so as to serve as its own support when rested atop the operating field.

Having described preferred embodiments of the invention with reference to the accompanying drawings, it is to be understood that the invention is not limited to those precise embodiments, and that various changes and modifications may be effected therein by one skilled in the art without departing from the scope or spirit of the invention.

What is claimed is:

1. A coupling attachment, comprising:
a position stabilization portion; and
a laparoscopic instrument restraint portion which is configured to include at least a structural portion which at least partially surrounds a shaft or one or more other portions of a laparoscopic instrument in a manner which positionally restrains or inhibits movement of the instrument and/or a portion or portions thereof when the position stabilization portion is supportably engaged.

2. A coupling attachment in combination with at least one laparoscopic instrument, the combination comprising:
the at least one laparoscopic instrument; and
the coupling attachment which includes:
a position stabilization portion; and
a laparoscopic instrument restraint portion which is configured to include at least a structural portion which at least partially surrounds a shaft or one or more other portions of said at least one laparoscopic instrument in a manner which positionally restrains or inhibits movement of the at least one laparoscopic instrument when the position stabilization portion is supportably engaged.

3. A method of positionally stabilizing a laparoscopic instrument, comprising:
removably engaging a portion of a laparoscopic instrument along a length thereof external of a patient with a coupling attachment which at least partially surrounds the portion of the laparoscopic instrument; and
supportably engaging the coupling attachment.
